(19) United States
(12) Patent Application Publication
(10) Pub. No.: US 2019/0151346 A1
(43) Pub. Date: May 23, 2019

(54) COMBINATIONS THERAPIES FOR THE TREATMENT OF CANCER

(71) Applicants: INSERM (INSTITUTE NATIONAL DE LA SANTÉ ET DE LA RECHERCHE MÉDICALE), Paris (FR); UNIVERSITE PARIS DIDEROT - PARIS 7, Paris (FR); OREGA BIOTECH, Ecully (FR); INSTITUT JEAN GODINOT, Reims (FR)

(72) Inventors: Reem AL-DACCAK, Paris (FR); Jérôme GIUSTINIANI, Sartrouville (FR); Jérémy BASTID, Francheville (FR); Armand BENSUSSAN, Paris (FR); Yacine MERROUCHE, Lyon (FR); Christian GARBAR, Bezannes (FR)

(21) Appl. No.: 16/300,233

(22) PCT Filed: May 9, 2017
(86) PCT No.: PCT/EP2017/061086

§ 371 Date: Nov. 9, 2018

(30) Foreign Application Priority Data
May 10, 2016 (EP) 16305540.3

(51) Int. Cl. A61K 31/713; C07K 16/24; C07K 16/28; A61P 35/00; C12N 15/113

(52) U.S. Cl. CPC A61K 31/713; C07K 16/244; A61K 45/06; A61P 35/00; C12N 15/113; C07K 16/2866

(57) ABSTRACT

The present invention relates to combinations therapies for the treatment of cancer. In particular the present invention relates to a method for enhancing the potency of an HER inhibitor administered to a patient as part of a treatment regimen, the method comprising administering to the patient a pharmaceutically effective amount of an IL-17B or IL-17E inhibitor in combination with the HER inhibitor.

### FIELD OF THE INVENTION

The present invention relates to combinations therapies for the treatment of cancer.

### BACKGROUND OF THE INVENTION

Over-expression of the epidermal growth factor receptor (EGFR) kinase is frequently associated with many cancers, including breast, lung, colorectal, head and neck cancers and is believed to contribute to the malignant growth of these tumors. Activation of EGFR stimulated signaling pathways promote multiple processes that are potentially cancer-promoting, e.g., proliferation, angiogenesis, cell motility and invasion, decreased apoptosis and induction of drug resistance. The development for use as anti-tumor agents of compounds that directly inhibit the kinase activity of the EGFR, as well as antibodies that reduce EGFR kinase activity by blocking EGFR activation, have been areas of intense research effort However resistance to said class of drugs has been observed. For example, estrogen receptor-, progesterone receptor- and HER2-negative breast cancer (Triple-Negative Breast Cancer (TNBC)) tumors have poor prognosis and are refractory to current therapeutics, including epidermal growth factor receptor (EGFR) inhibitors, with only 10-20% of patients with relevant clinical improvement. Resistance to anti-EGFR therapeutics is often associated with sustained phosphorylation of kinases promoting activation and translocation of EGFR to the nucleus, and/or with the inaccessibility of inhibitors to their target. The paracrine pathways that are active in TNBC microenvironment and can endorse these EGFR resistance-promoting events are not yet fully defined. Beside the EGFR, the receptor of IL-17B and a subunit of the IL-17E receptor (IL-17RB protein) is overexpressed in TNBC tumors and is associated to their bad prognosis (Mombelli S, Cochaud S, Merrouche Y, Garbar C, Antonicelli F, Laprevotte E, Alberici G, Bonnefoy N, Eliaou J F, Bastid J, Bensussan A, Giustiniani J. IL-17A and its homologs IL-25/IL-17E recruit the c-RAF/S6 kinase pathway and the generation of pro-oncogenic LMW-E in breast cancer cells. Sci Rep. 2015 Jul. 8; 5:11874.). The pro-inflammatory cytokine IL-17E is also abundant in TNBC tumors microenvironment and promotes their resistance to anti-mitotic therapies. IL-17E receptor signaling activates various cascades in breast cancer cells. Though, in the context of TNBC tumors resistance to anti-EGFR therapeutics the signaling cascades downstream IL-17E and IL-17-B receptor were never explored.

### SUMMARY OF THE INVENTION

The present invention relates to combinations therapies for the treatment of cancer. In particular, the present invention is defined by the claims.

### DETAILED DESCRIPTION OF THE INVENTION

Anti-epidermal growth factor receptor (EGFR), is an efficient therapeutic for various types of cancer. However, some tumors like TNBC are refractory to anti-EGFR despite the overexpression of the EGFR by these tumors. Furthering the understanding on the mechanisms of pathophysiology is therefore mandatory to develop more efficient strategies. The inventors have recently demonstrated that IL-17E, a member of the pro-inflammatory IL-17A family proteins, is abundant in the microenvironment of TNBC and induces their Docetaxel resistance (Mombelli S, et al. Sci Rep. 2015 Jul. 8; 5:11874). Similarly, IL-17B is expressed in breast cancer and promotes resistance to docetaxel (Emilie Laprevotte, et al. Interleukin-17B promotes chemoresistance of breast tumors through ERK1/2 anti-apoptotic pathway. [abstract]. In: Proceedings of the 106th Annual Meeting of the American Association for Cancer Research; 2015 Apr. 18-22; Philadelphia, Pa. Philadelphia (Pa.): AACR; Cancer Res 2015; 75(15 Suppl):Abstract nr 5027. doi:10.1158/1538-7445.AM2015-5027). IL-17A and its homologs IL-25/IL-17E recruit the c-RAF/S6 kinase pathway and the generation of pro-oncogenic LMW-E in breast cancer cells (Mombelli et al. Sci Rep. 2015 Jul. 8; 5:11874.). The expression of IL17-RB is also up-regulated and is associated with bad prognosis in breast cancer. Therefore, the inventors explored the link between IL-17B/E-IL-17RB axis and EGF signaling pathway in TNBC. They found that the engagement of IL-17E with its specific receptor IL-17RA/IL17RB induces the phosphorylation of PYK-2, Src kinases, and STAT3 and synergizes with EGF to induce Src-dependent transactivation of EGFR. Importantly, the combination of IL17E and EGF promoted the resistance of TBNC to the EGFR tyrosine kinase inhibitor Iressa, likely through the capacity of IL-17E to regulate the nuclear transport of pSTAT3 and pEGFR. Similar data were obtained using the IL-17B, demonstrating that it is involved in the same mechanism of resistance. Collectively, the data reveal the first evidence indicating the importance of IL-17B and IL-17E for resistance against anti-EGFR therapeutics and suggest blocking IL-17B or IL-17E or their receptor in combination with anti-EGFR as a novel bio-therapeutic strategy to combat cancer.

Accordingly, the first object of the present invention relates to a method for enhancing the potency of an HER inhibitor administered to a patient as part of a treatment regimen, the method comprising administering to the patient a pharmaceutically effective amount of an IL-17B or IL-17E inhibitor in combination with the HER inhibitor.

The second object of the present invention relates to a method of treating cancer in a patient in need thereof comprising administering to the patient a therapeutically effective combination of an HER inhibitor with an IL-17B or IL-17E inhibitor, wherein administration of the combination results in enhanced therapeutic efficacy relative to the administration of the HER inhibitor alone.

As used herein, the expression “enhancing the potency of an HER inhibitor” refers to the ability of the IL-17E or IL-17B inhibitor to increase the ability of the HER inhibitor to inhibit tumor cell growth

As used herein, the expression “enhanced therapeutic efficacy,” relative to cancer refers to a slowing or diminution of the growth of cancer cells or a solid tumor, or a reduction in the total number of cancer cells or total tumor burden. An “improved therapeutic outcome” or “enhanced therapeutic efficacy” therefore means there is an improvement in the condition of the patient according to any clinically acceptable criteria, including, for example, decreased tumor size, an increase in time to tumor progression, increased progression-free survival, increased overall survival time, an increase in life expectancy, or an improvement in quality of life. In particular, “improved” or “enhanced” refers to an improvement or enhancement of 1%, 5%, 10%, 25% 50%, 75%, 100%, or greater than 100% of any clinically acceptable indicator of therapeutic outcome or efficacy. As used herein, the expression “relative to” when used in the context of comparing the activity and/or efficacy of a combination composition comprising the HER inhibitor with the IL-17E or IL-17B inhibitor to the activity and/or efficacy of the HER inhibitor alone, refers to a comparison using amounts known to be comparable according to one of skill in the art.

As used herein, the term “cancer” has its general meaning in the art and includes, but is not limited to, solid tumors and blood-borne tumors. The term cancer includes diseases of the skin, tissues, organs, bone, cartilage, blood and vessels. The term “cancer” further encompasses both primary and metastatic cancers. Examples of cancers that may be treated by methods and compositions of the invention include, but are not limited to, cancer cells from the bladder, blood, bone, bone marrow, brain, breast, colon, esophagus, gastrointestinal tract, gum, head, kidney, liver, lung, nasopharynx, neck, ovary, prostate, skin, stomach, testis, tongue, or uterus. In addition, the cancer may specifically be of the following histological type, though it is not limited to these: neoplasm, malignant; carcinoma; undifferentiated carcinoma; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous; adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; Paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; and roblastoma, malignant; Sertoli cell carcinoma; Leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangio sarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; Ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

In some embodiments, the methods of the present invention are particularly suitable for the treatment of colorectal cancer, non-small cell lung carcinoma (NSCLC), adrenocortical carcinoma (ACC), pancreatic cancer, head and neck cancer, breast cancer (in particular triple negative breast cancer), or neuroblastoma.

In some embodiments, methods of the present invention are particularly suitable for the treatment of a cancer resistant to HER inhibitors or of a cancer resistant to EGFR (HER1) inhibitors. As used herein, the term “resistant” refers to the repeated outbreak of the cancer, or a progression of the cancer independently of whether the disease was cured before said outbreak or progression.

“Antineoplastic resistance” is the drug resistance of neoplastic (cancerous) cells, or the ability of cancer cells to survive and grow despite anti-cancer therapies. There are two general causes of antineoplastic therapy failure: Inherent properties, such as genetic characteristics, giving cancer cells their resistance, which is rooted in the concept of cancer cell heterogeneity and acquired resistance after drug exposure. Cancer cells can become resistant to drugs by various mechanisms, including: altered membrane transport, enhanced DNA repair, apoptotic pathway defects, alteration of target molecules, protein and pathway mechanisms, such as enzymatic deactivation. Since cancer is a genetic disease, two genomic events underlie these mechanisms of acquired drug resistance: Genome alterations (e.g. gene amplification and deletion) and epigenetic modifications (Housman et al., Cancer, 2014, 6, 1769-1792). Possible mechanisms of tumor resistance to EFGR-targeted therapies are e.g. disclosed in Hoppoer-Borge et al. (Expert Opin Ther Tragets. 2009 March; 13(3):339-362).

As used therein, the expression “cancer resistant to HER inhibitors” or a “cancer resistant to EGFR (HER1) inhibitors” refers to the fact that the majority of the cancer patients do not respond to these treatments (HER or EGFR inhibitors) and/or have a poor prognostic.

As used herein, the term “treatment” or “treat” refer to both prophylactic or preventive treatment as well as curative or disease modifying treatment, including treatment of patient at risk of contracting the disease or suspected to have contracted the disease as well as patients who are ill or have been diagnosed as suffering from a disease or medical condition, and includes suppression of clinical relapse. The treatment may be administered to a patient having a medical disorder or who ultimately may acquire the disorder, in order to prevent, cure, delay the onset of, reduce the severity of, or ameliorate one or more symptoms of a disorder or recurring disorder, or in order to prolong the survival of a patient beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., pain, disease manifestation, etc.]).

As used herein the “HER” has its general meaning in the art and refers to a receptor protein tyrosine kinase which belongs to the HER receptor family and includes EGFR, HER2, HER3 and HER4 receptors. As used herein the terms “ErbB1,” “HER1”, “epidermal growth factor receptor” and “EGFR” are used interchangeably herein and refer to EGFR as disclosed, for example, in Carpenter et al. Ann. Rev. Biochem. 56:881-914 (1987), As used herein, the terms “ErbB2” and “HER2” are used interchangeably herein and refer to human HER2 protein described, for example, in Semba et al, PNAS (USA) 82:6497-6501 (1985) and Yamamoto et al. Nature 319:230-234 (1986) (Genebank accession number X03363). As used herein, the term “ErbB3” and “HER3” refer to the receptor polypeptide as disclosed, for example, in U.S. Pat. Nos. 5,183,884 and 5,480,968 as well as Kraus et al. PNAS (USA) 86:9193-9197 (1989). As used herein, the terms “ErbB4” and “HER4” refer to the receptor polypeptide as disclosed, for example, in EP Pat Appln No 599,274; Plowman et al, Proc. Natl. Acad. Sci. USA, 90: 1746-1750 (1993); and Plowman et al, Nature, 366:473-475 (1993). By “HER ligand” is meant a polypeptide which binds to and/or activates an HER receptor.

As used herein the term “HER inhibitor” refers to an agent which interferes with HER activation or function. Examples of HER inhibitors include HER antibodies (e.g. EGFR, HER2, HER3, or HER4 antibodies); small organic molecule HER antagonists; HER tyrosine kinase inhibitors; HER2 and EGFR dual tyrosine kinase inhibitors such as lapatinib/GW572016; antisense molecules (see, for example, WO2004/87207); and/or agents that bind to, or interfere with function of, downstream signaling molecules, such as MAPK or Akt. Typically, the HER inhibitor is an antibody or small organic molecule which binds to an HER receptor.

In some embodiments, the HER inhibitor is a “HER dimerization inhibitor” which is an agent which inhibits formation of an HER dimer or HER heterodimer.

In some embodiments, the HER inhibitor is a small organic molecule. As used herein, the term “small organic molecule” refers to a molecule of size comparable to those organic molecules generally sued in pharmaceuticals. The term excludes biological macromolecules (e.g.; proteins, nucleic acids, etc.); preferred small organic molecules range in size up to 2000 Da, and most preferably up to about 1000 Da.

In some embodiments, the HER inhibitor is a tyrosine kinase inhibitor. A “tyrosine kinase inhibitor” is a molecule which inhibits tyrosine kinase activity of the HER receptor. Examples of such inhibitors include the small organic molecule HER2 tyrosine kinase inhibitor such as TAK165 available from Takeda; CP-724,714, an oral selective inhibitor of the ErbB2 receptor tyrosine kinase (Pfizer and OSI); dual-HER inhibitors such as EKB-569 (available from Wyeth) which preferentially binds EGFR but inhibits both HER2 and EGFR-overexpressing cells; GW572016 (available from Glaxo) an oral HER2 and EGFR tyrosine kinase inhibitor; PKI-166 (available from Novartis); pan-HER inhibitors such as Canertinib (CI-1033; Pharmacia); non selective HER inhibitors such as Imatinib mesylate (Gleevec™); MAPK extracellular regulated kinase I inhibitor CI-1040 (available from Pharmacia); quinazolines, such as PD 153035, 4-(3-chloroanilino) quinazoline; pyridopyrimidines; pyrimidopyrimidines; pyrrolopyrimidines, such as CGP 59326, CGP 60261 and CGP 62706; pyrazolopyrimidines, 4-(phenylamino)-7H-pyrrolo [2,3-d]pyrimidines; curcumin (diferuloyl methane, 4,5-bis(4-fluoroanilino)phthalimide); tyrphostines containing nitrothiophene moieties; PD-0183805 (Warner-Lamber); quinoxalines (U.S. Pat. No. 5,804,396); tryphostins (U.S. Pat. No. 5,804,396); ZD6474 (Astra Zeneca); PTK-787 (Novartis/Schering AG); pan-HER inhibitors such as CI-1033 (Pfizer); PKI 166 (Novartis); GW2016 (Glaxo SmithKline); CI-1033 (Pfizer); EKB-569 (Wyeth); Semaxinib (Sugen); ZD6474 (AstraZeneca); PTK-787 (Novartis/Schering AG); INC-1C1 (Imclone); or as described in any of the following patent publications: U.S. Pat. No. 5,804,396; WO99/09016 (American Cyanimid); WO98/43960 (American Cyanamid); WO97/38983 (Warner Lambert); WO99/06378 (Warner Lambert); WO99/06396 (Warner Lambert); WO96/30347 (Pfizer, Inc); WO96/33978 (Zeneca); WO96/3397 (Zeneca); and WO96/33980 (Zeneca).

In some embodiments, the HER inhibitor is an EGFR inhibitor. EGFR inhibitors are well known in the art (Inhibitors of erbB-1 kinase; Expert Opinion on Therapeutic Patents December 2002, Vol. 12, No. 12, Pages 1903-1907, Susan E Kane. Cancer therapies targeted to the epidermal growth factor receptor and its family members. Expert Opinion on Therapeutic Patents February 2006, Vol. 16, No. 2, Pages 147-164. Peter Traxler Tyrosine kinase inhibitors in cancer treatment (Part II). Expert Opinion on Therapeutic Patents December 1998, Vol. 8, No. 12, Pages 1599-1625). Examples of such agents include antibodies and small organic molecules that bind to EGFR. Examples of antibodies which bind to EGFR include MAb 579 (ATCC CRL HB 8506), MAb 455 (ATCC CRL HB8507), MAb 225 (ATCC CRL 8508), MAb 528 (ATCC CRL 8509) (see, U.S. Pat. No. 4,943,533, Mendelsohn et al.) and variants thereof, such as chimerized 225 (C225 or Cetuximab; ERBUTIX®) and reshaped human 225 (H225) (see, WO 96/40210, Imclone Systems Inc.); IMC-1 1F8, a fully human, EGFR-targeted antibody (Imclone); antibodies that bind type II mutant EGFR (U.S. Pat. No. 5,212,290); humanized and chimeric antibodies that bind EGFR as described in U.S. Pat. No. 5,891,996; and human antibodies that bind EGFR, such as ABX-EGF (see WO98/50433, Abgenix); EMD 55900 (Stragliotto et al. Eur. J. Cancer 32A:636-640 (1996)); EMD7200 (matuzumab) a humanized EGFR antibody directed against EGFR that competes with both EGF and TGF-alpha for EGFR binding; and mAb 806 or humanized mAb 806 (Johns et al, J. Biol. Chem. 279(29):30375-30384 (2004)). The anti-EGFR antibody may be conjugated with a cytotoxic agent, thus generating an immunoconjugate (see, e.g., EP659,439A2, Merck Patent GmbH). Examples of small organic molecules that bind to EGFR include ZD 1839 or Gefitinib (IRESSA™; Astra Zeneca); CP-358774 or erlotinib (TARCEVA™; Genentech/OSI); and AG1478, AG1571 (SU 5271; Sugen); EMD-7200. In some embodiments, the HER inhibitor is a small organic molecule pan-HER inhibitor such as dacomitinib (PF-00299804).

In some embodiments, the HER inhibitor is an “anti-HER antibody” which is an antibody that binds to an HER receptor. Patent publications related to HER antibodies include: U.S. Pat. Nos. 5,677,171, 5,720,937, 5,720,954, 5,725,856, 5,770,195, 5,772,997, 6,165,464, 6,387,371, 6,399,063, US2002/0192211A1, U.S. Pat. Nos. 6,015,567, 6,333,169, 4,968,603, 5,821,337, U.S. Pat. Nos. 6,054,297, 6,407,213, 6,719,971, 6,800,738, US2004/0236078A1, U.S. Pat. Nos. 5,648,237, 6,267,958, 6,685,940, 6,821,515, WO98/17797, U.S. Pat. Nos. 6,127,526, 6,333,398, 6,797,814, 6,339,142, 6,417,335, 6,489,447, WO99/31140, US2003/0147884A1, US2003/0170234A1, US2005/0002928A1, U.S. Pat. No. 6,573,043, US2003/0152987A1, WO99/48527, US2002/0141993A1, WO01/00245, US2003/0086924, US2004/0013667A1, WO00/69460, WO01/00238, WO01/15730, U.S. Pat. No. 6,627,196B1, U.S. Pat. No. 6,632,979B1, WO01/00244, US2002/0090662A1, WO01/89566, US2002/0064785, US2003/0134344, WO04/24866, US2004/0082047, US2003/0175845A1, WO03/087131, US2003/0228663, WO2004/008099A2, US2004/0106161, WO2004/048525, US2004/0258685A1, U.S. Pat. Nos. 5,985,553, 5,747,261, 4,935,341, 5,401,638, 5,604,107, WO 87/07646, WO 89/10412, WO 91/05264, EP 412,116 B1, EP 494,135 B1, U.S. Pat. No. 5,824,311, EP 444,181 B1, EP 1,006,194 A2, US 2002/0155527A1, WO 91/02062, U.S. Pat. Nos. 5,571,894, 5,939,531, EP 502,812 B1, WO 93/03741, EP 554,441 B1, EP 656,367 A1, U.S. Pat. Nos. 5,288,477, 5,514,554, 5,587,458, WO 93/12220, WO 93/16185, U.S. Pat. No. 5,877,305, WO 93/21319, WO 93/21232, U.S. Pat. No. 5,856,089, WO 94/22478, U.S. Pat. Nos. 5,910,486, 6,028,059, WO 96/07321, U.S. Pat. Nos. 5,804,396, 5,846,749, EP 711,565, WO 96/16673, U.S. Pat. Nos. 5,783,404, 5,977,322, 6,512,097, WO 97/00271, U.S. Pat. Nos. 6,270,765, 6,395,272, 5,837,243, WO 96/40789, U.S. Pat. Nos. 5,783,186, 6,458,356, WO 97/20858, WO 97/38731, U.S. Pat. Nos. 6,214,388, 5,925,519, WO 98/02463, U.S. Pat. No. 5,922,845, WO 98/18489, WO 98/33914, U.S. Pat. No. 5,994,071, WO 98/45479, U.S. Pat. No. 6,358,682 B1, US 2003/0059790, WO 99/55367, WO 01/20033, US 2002/0076695 A1, WO 00/78347, WO 01/09187, WO 01/21192, WO 01/32155, WO 01/53354, WO 01/56604, WO 01/76630, WO02/05791, WO 02/11677, U.S. Pat. No. 6,582,919, US2002/0192652A1, US 2003/0211530A1, WO 02/44413, US 2002/0142328, U.S. Pat. No. 6,602,670 B2, WO 02/45653, WO 02/055106, US 2003/0152572, US 2003/0165840, WO 02/087619, WO 03/006509, WO03/012072, WO 03/028638, US 2003/0068318, WO 03/041736, EP 1,357,132, US 2003/0202973, US 2004/0138160, U.S. Pat. Nos. 5,705,157, 6,123,939, EP 616,812 B1, US 2003/0103973, US 2003/0108545, U.S. Pat. No. 6,403,630 B1, WO 00/61145, WO 00/61185, U.S. Pat. No. 6,333,348 B1, WO 01/05425, WO 01/64246, US 2003/0022918, US 2002/0051785 A1, U.S. Pat. No. 6,767,541, WO 01/76586, US 2003/0144252, WO 01/87336, US 2002/0031515 A1, WO 01/87334, WO 02/05791, WO 02/09754, US 2003/0157097, US 2002/0076408, WO 02/055106, WO 02/070008, WO 02/089842, WO 03/86467, WO2013164689, and WO2012059857.

In some embodiments, the HER inhibitor is selected from the group consisting of cetuximab, panitumumab (Vectibix™), zalutumumab (HuMax-EGFR), nimotuzumab (h-R3, BIOMAb EGFR, TheraCIM, Theraloc), erlotinib (OSI-744, Tarceva), gefitinib (ZD1839, Irissa), lapatinib (Tykerb), lapatinib ditosylate (GW-572016, Tyverb), neratinib (HKI-272), canertinib (CI-1033), vandetanib (Caprelsa), afatinib (BIBW2992, Gilotrif or Giotrif), TAK-285 (dual HER2 and EGFR inhibitor), Varlitinib (ARRY334543) (dual HER2 and EGFR inhibitor), Dacomitinib (PF299804, PF299) (pan-ErbB inhibitor), OSI-420 (Desmethyl Erlotinib) (EGFR inhibitor), Sapitinib (AZD8931) (EGFR, HER2 and HER3 inhibitor), AEE788 (NVP-AEE788) (EGFR, HER2 and VEGFR 1 12 inhibitor), Pelitinib (EKB-569) (pan-ErbB inhibitor), CUDC-101 (EGFR, HER2 and HDAC inhibitor), XL647 (dual HER2 and EGFR inhibitor), BMS-599626 (AC480) (dual HER2 and EGFR inhibitor), Midostaurin (PKC412) (EGFR, PKC, cyclic AMP-dependent protein kinase and S6 kinase inhibitor), Falnidamol (BIBX1382) (EGFR inhibitor) and AP261 13 (ALK and EGFR inhibitor). The inhibitors cetuximab, panitumumab, zalutumumab, nimotuzumab are monoclonal antibodies, erlotinib, gefitinib, lapatinib, neratinib, canertinib, vandetanib and afatinib are tyrosine kinase inhibitors. Without being an exhaustive list, other useful inhibitors are selected from the group consisting of Nazartinib (EGF816, NVS-816), Naquotinib (ASP8273), Olmutinib (HM61713, BI 1482694), AG-490 (Tyrphostin B42), WZ4002, AG-1478 (Tyrphostin AG-1478), PD 153035, WZ3146, WZ8040, AST-1306, Rociletinib (CO-1686, AVL-301), Icotinib, WHI-P154, Daphnetin, PD168393, Tyrphostin 9, CNX-2006, AG-18, AZ5104, Osimertinib (AZD9291), CL-387785 (EKI-785), (−)-Epigallocatechin Gallate, AZD3759, Poziotinib (HM781-36B), Chrysophanic Acid, Butein, AG-494, Compound 56, DAPH, Erbstatin, Lavendustin A, Lavendustin C methyl ester, PD174265, SU 4984, Tyrphostin 25 (RG-50875), Tyrphostin 23 (RG-50810), Tyrphostin 47 (RG-50864) (AG-213), Tyrphostin 51.

The interleukin 17 (IL-17) family comprises 6 interleukins (IL-17A, IL-17B, IL-17C, IL-17D, IL-17E and IL-17F) and their receptors (IL-17RA, IL-17RB, IL-17RC, IL-17RD and IL-17RE) (Gaffen, S. L. (2009) “Structure and signalling in the IL-17 receptor family” Nature reviews. Immunology 9(8): 556-567). IL-17B binds the dimeric IL-17RB receptor and IL-17E binds a complex of IL-17RA and IL-17RB.

As used herein the term “IL-17E” has its general meaning in the art and a polypeptide having a sequence according to GenBank Acc. No. N073626 or NP758525, the product of the human IL-17E gene, and include all of the variants, isoforms or species homologs of IL-17E. The interleukin is also named IL-25. The term “IL-17E receptor” as used herein means a receptor or a receptor complex mediating IL-17E signaling. IL-17E signaling requires two receptors, IL17RB and IL17RA, which may form a heteromeric complex. IL-17E binds to IL17RB with high affinity, whereas IL17RA does not bind IL-17E but is required for activating signaling pathways upon ligand binding (Rickel et al., J. Immunology 181:4299-310, 2008). Thus, “IL-17E receptor” contemplates both IL17RB and IL17RA. As used herein, the term “IL-17E signaling” as used herein means the processes initiated by IL-17E or a second IL-17E receptor ligand interacting with the IL-17E receptor on the cell surface, resulting in measurable changes in cell function. IL-17E receptor complex includes IL7RB and IL17RA, and ligand binding activates downstream signal transduction pathways. Typically, IL-17E signaling can be assessed by functional assays measuring for example effect of IL-17E receptor ligand on cell proliferation or differentiation, or using reporter genes and reporter gene constructs.

As used herein the term “IL-17B” has its general meaning in the art and a polypeptide having a sequence according to GenBank Acc. No. NP_001304916.1 or NP 055258.1, the product of the human IL-17B gene, and include all of the variants, isoforms or species homologs of IL-17B. As used herein, the term “IL-17B signaling” as used herein means the processes initiated by IL-17B or a second IL-17B receptor ligand interacting with the IL-17RB receptor on the cell surface, resulting in measurable changes in cell function. Typically, IL-17B signaling can be assessed by functional assays measuring for example effect of IL-17B receptor ligand on cell proliferation or differentiation, or using reporter genes and reporter gene constructs.

As used herein, the term “IL17RB” (IL-17BR, CRL4, EVI27, IL17RH1, or MGC5245) as used herein means “interleukin 17 receptor B”, a polypeptide having an amino acid sequence according to GenBank Acc. No. NP061195, the product of the human IL17RB receptor gene, and include all of the variants, isoforms and species homologs of IL17RB. Both IL-17E and IL-17B are ligands for IL17RB, but the receptor binds IL-17E with higher affinity (Lee, et al., J. Biol. Chem. 276, 1660-64, 2001).

As used herein, the term “IL17RA”, (CD217, IL17R, CDw217, IL-17RA, hIL-17R, or MGC10262) as used herein means “interleukin 17 receptor A”, a polypeptide having an amino acid sequence according to GenBank Ace. No. NP055154, the product of the human IL17RA receptor gene, and include all of the variants, isoforms and species homologs of IL17RA. Variants of IL17RB and IL17RA also include soluble mature receptors.

Accordingly, as used herein the terms “IL-17E inhibitor” and “IL-17B inhibitors” refers to any compound that is able to inhibit the IL-17E and IL-17B signalling respectively. The IL-17E or IL-17B inhibitor to be used in the methods described herein is a molecule that blocks, suppresses, or reduces (including significantly) the biological activity of the IL-17E or IL-17B cytokine, including downstream pathways mediated by IL-17E or IL-17B signaling. Thus the term “IL17-E inhibitor” or “IL-17B inhibitor” implies no specific mechanism of biological action whatsoever, and is deemed to expressly include and encompass all possible pharmacological, physiological, and biochemical interactions with IL-17E or IL-17B whether direct or indirect.

In some embodiments, the IL-17E inhibitor is selected from the group consisting of antibodies directed against IL-17E and antibodies directed against a receptor of a IL-17E (e.g., an antibody specifically binds IL17RA or IL17RB or the dimeric complex formed thereby).

In some embodiments, the IL-17B inhibitor is selected from the group consisting of antibodies directed against IL-17B and antibodies directed against a receptor of a IL-17B (e.g., an antibody specifically binds IL17RB or the dimeric complex formed thereby).

As used herein, the term “antibody” is thus used to refer to any antibody-like molecule that has an antigen binding region, and this term includes antibody fragments that comprise an antigen binding domain such as Fab′, Fab, F(ab′)2, single domain antibodies (DABs), TandAbs dimer, Fv, scFv (single chain Fv), dsFv, ds-scFv, Fd, linear antibodies, minibodies, diabodies, bispecific antibody fragments, bibody, tribody (scFv-Fab fusions, bispecific or trispecific, respectively); sc-diabody; kappa(lamda) bodies (scFv-CL fusions); BiTE (Bispecific T-cell Engager, scFv-scFv tandems to attract T cells); DVD-Ig (dual variable domain antibody, bispecific format); SIP (small immunoprotein, a kind of minibody); SMIP (“small modular immunopharmaceutical” scFv-Fc dimer; DART (ds-stabilized diabody “Dual Affinity ReTargeting”); small antibody mimetics comprising one or more CDRs and the like. The techniques for preparing and using various antibody-based constructs and fragments are well known in the art (see Kabat et al., 1991, specifically incorporated herein by reference). Diabodies, in particular, are further described in EP 404, 097 and WO 93/1 1 161; whereas linear antibodies are further described in Zapata et al. (1995). Antibodies can be fragmented using conventional techniques. For example, F(ab′)2 fragments can be generated by treating the antibody with pepsin. The resulting F(ab′)2 fragment can be treated to reduce disulfide bridges to produce Fab′ fragments. Papain digestion can lead to the formation of Fab fragments. Fab, Fab′ and F(ab′)2, scFv, Fv, dsFv, Fd, dAbs, TandAbs, ds-scFv, dimers, minibodies, diabodies, bispecific antibody fragments and other fragments can also be synthesized by recombinant techniques or can be chemically synthesized. Techniques for producing antibody fragments are well known and described in the art. For example, each of Beckman et al., 2006; Holliger & Hudson, 2005; Le Gall et al., 2004; Reff & Heard, 2001; Reiter et al., 1996; and Young et al., 1995 further describe and enable the production of effective antibody fragments. In some embodiments, the antibody of the present invention is a single chain antibody. As used herein the term “single domain antibody” has its general meaning in the art and refers to the single heavy chain variable domain of antibodies of the type that can be found in Camelid mammals which are naturally devoid of light chains. Such single domain antibody are also “Nanobody®”. For a general description of (single) domain antibodies, reference is also made to the prior art cited above, as well as to EP 0 368 684, Ward et al. (Nature 1989 Oct. 12; 341 (6242): 544-6), Holt et al., Trends Biotechnol., 2003, 21(11):484-490; and WO 06/030220, WO 06/003388.

In some embodiments, the antibody is a humanized antibody. As used herein, “humanized” describes antibodies wherein some, most or all of the amino acids outside the CDR regions are replaced with corresponding amino acids derived from human immunoglobulin molecules. Methods of humanization include, but are not limited to, those described in U.S. Pat. Nos. 4,816,567, 5,225,539, 5,585,089, 5,693,761, 5,693,762 and 5,859,205, which are hereby incorporated by reference.

In some embodiments, the antibody is a fully human antibody. Fully human monoclonal antibodies also can be prepared by immunizing mice transgenic for large portions of human immunoglobulin heavy and light chain loci. See, e.g., U.S. Pat. Nos. 5,591,669, 5,598,369, 5,545,806, 5,545,807, 6,150,584, and references cited therein, the contents of which are incorporated herein by reference. These animals have been genetically modified such that there is a functional deletion in the production of endogenous (e.g., murine) antibodies. The animals are further modified to contain all or a portion of the human germ-line immunoglobulin gene locus such that immunization of these animals will result in the production of fully human antibodies to the antigen of interest. Following immunization of these mice (e.g., XenoMouse (Abgenix), HuMAb mice (Medarex/GenPharm)), monoclonal antibodies can be prepared according to standard hybridoma technology. These monoclonal antibodies will have human immunoglobulin amino acid sequences and therefore will not provoke human anti-mouse antibody (KAMA) responses when administered to humans. In vitro methods also exist for producing human antibodies. These include phage display technology (U.S. Pat. Nos. 5,565,332 and 5,573,905) and in vitro stimulation of human B cells (U.S. Pat. Nos. 5,229,275 and 5,567,610). The contents of these patents are incorporated herein by reference.

In some embodiments, the antibody does not comprise an Fc portion that induces antibody dependent cellular cytotoxicity (ADCC). The terms “Fc domain,” “Fc portion,” and “Fc region” refer to a C-terminal fragment of an antibody heavy chain, e.g., from about amino acid (aa) 230 to about aa 450 of human gamma heavy chain or its counterpart sequence in other types of antibody heavy chains (e.g., α, δ, ε and μ for human antibodies), or a naturally occurring allotype thereof. Unless otherwise specified, the commonly accepted Kabat amino acid numbering for immunoglobulins is used throughout this disclosure (see Kabat et al. (1991) Sequences of Protein of Immunological Interest, 5th ed., United States Public Health Service, National Institute of Health, Bethesda, Md.). In some embodiments, the antibody of the present invention does not comprise an Fc domain capable of substantially binding to a FcgRIIIA (CD16) polypeptide. In some embodiments, the antibody of the present invention lacks an Fc domain (e.g. lacks a CH2 and/or CH3 domain) or comprises an Fc domain of IgG2 or IgG4 isotype. In some embodiments, the antibody of the present invention consists of or comprises a Fab, Fab′, Fab′-SH, F (ab′) 2, Fv, a diabody, single-chain antibody fragment, or a multispecific antibody comprising multiple different antibody fragments. In some embodiments, the antibody of the present invention is not linked to a toxic moiety. In some embodiments, one or more amino acids selected from amino acid residues can be replaced with a different amino acid residue such that the antibody has altered C2q binding and/or reduced or abolished complement dependent cytotoxicity (CDC). This approach is described in further detail in U.S. Pat. No. 6,194,551.

In some embodiments, the HER, IL-17E or IL-17B inhibitor is an inhibitor of HER, IL-17E, IL-17B or IL-17RB expression. An “inhibitor of expression” refers to a natural or synthetic compound that has a biological effect to inhibit the expression of a gene. In a preferred embodiment of the invention, said inhibitor of gene expression is a siRNA, an antisense oligonucleotide or a ribozyme. For example, anti-sense oligonucleotides, including anti-sense RNA molecules and anti-sense DNA molecules, would act to directly block the translation of HER, IL-17E or IL-17B mRNA by binding thereto and thus preventing protein translation or increasing mRNA degradation, thus decreasing the level of HER, IL-17E or IL-17B, and thus activity, in a cell. For example, antisense oligonucleotides of at least about 15 bases and complementary to unique regions of the mRNA transcript sequence encoding HER, IL-17E or IL-17B can be synthesized, e.g., by conventional phosphodiester techniques. Methods for using antisense techniques for specifically inhibiting gene expression of genes whose sequence is known are well known in the art (e.g. see U.S. Pat. Nos. 6,566,135; 6,566,131; 6,365,354; 6,410,323; 6,107,091; 6,046,321; and 5,981,732). Small inhibitory RNAs (siRNAs) can also function as inhibitors of expression for use in the present invention. HER, IL-17E or IL-17B gene expression can be reduced by contacting a patient or cell with a small double stranded RNA (dsRNA), or a vector or construct causing the production of a small double stranded RNA, such that HER, IL-17E or IL-17B gene expression is specifically inhibited (i.e. RNA interference or RNAi). Antisense oligonucleotides, siRNAs, shRNAs and ribozymes of the invention may be delivered in vivo alone or in association with a vector. In its broadest sense, a “vector” is any vehicle capable of facilitating the transfer of the antisense oligonucleotide, siRNA, shRNA or ribozyme nucleic acid to the cells and typically cells expressing HER, IL-17E or IL-17B. Typically, the vector transports the nucleic acid to cells with reduced degradation relative to the extent of degradation that would result in the absence of the vector. In general, the vectors useful in the invention include, but are not limited to, plasmids, phagemids, viruses, other vehicles derived from viral or bacterial sources that have been manipulated by the insertion or incorporation of the antisense oligonucleotide, siRNA, shRNA or ribozyme nucleic acid sequences. Viral vectors are a preferred type of vector and include, but are not limited to nucleic acid sequences from the following viruses: retrovirus, such as moloney murine leukemia virus, harvey murine sarcoma virus, murine mammary tumor virus, and rous sarcoma virus; adenovirus, adeno-associated virus; SV40-type viruses; polyoma viruses; Epstein-Barr viruses; papilloma viruses; herpes virus; vaccinia virus; polio virus; and RNA virus such as a retrovirus. One can readily employ other vectors not named but known to the art.

As used herein the term “co-administering” as used herein means a process whereby the combination of the IL-17E or IL-17B inhibitor and the HER inhibitor, is administered to the same patient. The IL-17E or IL-17B inhibitor and the HER inhibitor may be administered simultaneously, at essentially the same time, or sequentially. The IL-17E or IL-17B inhibitor and the HER inhibitor need not be administered by means of the same vehicle. The IL-17E or IL-17B inhibitor and the HER inhibitor may be administered one or more times and the number of administrations of each component of the combination may be the same or different. In addition, the IL-17E or IL-17B inhibitor and the HER inhibitor need not be administered at the same site.

As used herein, the term “therapeutically effective combination” as used herein refers to an amount or dose of an IL-17E inhibitor together with the amount or dose of the HER inhibitor that is sufficient to treat the cancer. The amount of the IL-17E or IL-17B inhibitor in a given therapeutically effective combination may be different for different individuals and different tumor types, and will be dependent upon the one or more additional agents or treatments included in the combination. The “therapeutically effective amount” is determined using procedures routinely employed by those of skill in the art such that an “improved therapeutic outcome” results. It will be understood, however, that the total daily usage of the compounds and compositions of the present invention will be decided by the attending physician within the scope of sound medical judgment. The specific therapeutically effective dose level for any particular patient will depend upon a variety of factors including the disorder being treated and the severity of the disorder; activity of the specific compound employed; the specific composition employed, the age, body weight, general health, sex and diet of the patient; the time of administration, route of administration, and rate of excretion of the specific compound employed; the duration of the treatment; drugs used in combination or coincidental with the specific polypeptide employed; and like factors well known in the medical arts. For example, it is well within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Typically, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the patient to be treated. A medicament typically contains from about 0.01 mg to about 500 mg of the active ingredient, preferably from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

According to the invention, the IL-17E or IL-17B inhibitor and the HER inhibitor are administered to the patient in the form of a pharmaceutical composition. Typically, the IL-17E or IL-17B inhibitor and the HER inhibitor may be combined with pharmaceutically acceptable excipients, and optionally sustained-release matrices, such as biodegradable polymers, to form therapeutic compositions. “Pharmaceutically” or “pharmaceutically acceptable” refer to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type. In the pharmaceutical compositions of the present invention for oral, sublingual, subcutaneous, intramuscular, intravenous, transdermal, local or rectal administration, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms. Typically, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions. The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. Solutions comprising compounds of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms. The IL-17E or IL-17B inhibitor and the HER inhibitor can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin. Sterile injectable solutions are prepared by incorporating the active compounds in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the typical methods of preparation are vacuum-drying and freeze-drying techniques which yield a powder of the active ingredient plus any additional desired ingredient from a previously sterile-filtered solution thereof. The preparation of more, or highly concentrated solutions for direct injection is also contemplated, where the use of DMSO as solvent is envisioned to result in extremely rapid penetration, delivering high concentrations of the active agents to a small tumor area. Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. Some variation in dosage will necessarily occur depending on the condition of the patient being treated. The person responsible for administration will, in any event, determine the appropriate dose for the individual patient.

The invention will be further illustrated by the following figures and examples. However, these examples and figures should not be interpreted in any way as limiting the scope of the present invention.

### FIGURES

FIG. 1. IL-17E phosphorylates EGFR and kinases essential for its activation. IJG-1731 (A), BT20 (B), and MDA-MB468 (C) cells were cultured alone or in the presence of IL-17E (long/ml) or EGF (10 ng/ml), then the phosphorylation of EGFR at residues Y845 and Y1086, of STAT3 at Y705, PYK-2 at Y402 and Src at Y416 was assessed by Western blotting. Membranes were re-blotted with anti-EGF or anti-STAT3α/β antibodies as control of equal loading. Data are representative of 3 independent experiments.

FIG. 2. IL-17E-induced EGFR phosphorylation depends on Src and EGFR kinase activities. IJG-1731, BT20, and MDA-MB468 cells were treated with the Src specific inhibitor AZM475271 (1004) (A), Iressa (0.2504) (B), or with control DMSO then stimulated with IL-17E (10 ng/ml) or EGF (10 ng/ml) or with medium alone. Phosphorylation of EGFR and Src was then assessed by Western blotting using specific antibodies. Loading control was ascertained by re-blotting the membranes with an anti-EGFR antibody. Data are representative of at least 2 independent experiments.

FIG. 3. IL-17E synergizes with EGF to phosphorylate Src kinase. MDA-MB468 cells were left untreated or treated with blocking anti-IL-17RB mAb (10 μg/ml) or its istotype IgG control then stimulated with IL-17E (1 ng/ml) alone or in combination with various concentrations of EGF (0.1-10 ng/ml). The Phosphorylation of Src (p416Src) was then assessed by Western blotting using specific anti-pSrc. Re-blotting with anti-Src antibody ascertained equal loading. Data are representative of 2 independent experiments.

FIG. 4. IL-17E co-translocates pEGFR and pSTAT3 to the nucleus. MDA-MB468 (A) or BT20 (B) cells were stimulated with IL-17E (10 ng/ml), EGF (long/ml) or a combination of both. In the upper panel of (A) and (B), localization of EGFR as assessed by immunostaining with anti-EGFR antibody (red). Nuclei were visualized with Dapi (blue). In the lower panel of (A) and (B), the translocation of EGFR, STAT3α/β, and their phosphorylated counterparts from the cytoplasm to the nucleus as assessed by Western blotting using specific antibodies. Anti-β actin and H3 histone antibodies were used as loading controls for cytoplasmic and nuclear fractions, respectively. Data are representative of 2 independent experiments.

FIG. 5. IL-17-A, -B or -E phosphorylates EGFR and essentials kinases, alone or and in synergy with EGF. MDA-MB468 (A), and IJG-1731 (B) cells were cultured alone or in the presence of IL-17A, IL-17B or IL-17E (10 ng/ml) with or without EGF (10 ng/ml), then the phosphorylation of EGFR at residue Y1086, of STAT3 at Y705, PYK-2 at Y402 and Src at Y416 was assessed by Western blotting. Membranes were re-blotted with anti-EGF or anti-STAT3a/b antibodies as control of equal loading. Data are representative of 2 independent experiments.

FIG. 6. IL-17B or -E-induced EGFR phosphorylation depends on Src kinase activity. MDA-MB468 and BT-20 cells were treated with the Src specific inhibitor AZM475271 (10 μM) (or with control DMSO) then stimulated with IL-17B, -E, EGF (or with medium alone). Phosphorylation of EGFR and Src was then assessed by Western blotting using specific antibodies. Loading control was ascertained by re-blotting the membranes with an anti-EGFR antibody. Data are representative of at least 2 independent experiments.

FIG. 7. IL-17B translocates EGFR. MDA-MB468 cells were stimulated with IL-17B (10 ng/ml), EGF (10 ng/ml) or a combination of both. localization of EGFR as assessed by immunostaining with anti-EGFR antibody. Nuclei were visualized with Dapi.

### EXAMPLE

Materials and Methods

Cell Culture

BT20 and MDA-MB468 triple negative (HER2-, ER-, PR-) were obtained from American Type Culture Collection (ATCC N°HTB19, and ATCC N°HTB132, respectively). The LumB, and Her2-, ER-, PR-negative IJG-1731 cell line was previously established at the laboratory as described (Mombelli et al. Sci Rep. 2015 Jul. 8; 5:11874.) BT20 and IJG-1731 cells were grown in complete RPMI-1640 media with L-glutamine, supplemented with 10% fetal calf serum (FCS) and penicillin—streptomycin solution (100 μg/ml each) (Life technology, Saint-Aubain, France). MDA-MB468 cells were grown in a complete DMEM-F12 media with glutamine, 10% FCS and penicillin—streptomycin. All cells were maintained in a humidified 5% CO2 atmosphere at 37° C. All experiments were conducted with cells at confluency and starved for an overnight.

Antibodies and Reagents

Rabbit anti-pEGFR (Y845), anti-pEGFR (Y1086), anti-pPYK2 (Y402), anti-pSTAT3 (Y705), anti-pSrc Family (Y416), anti-EGFR, anti-STAT3, anti-β-actin and Alexa 594-conjugated anti-rabbit F(ab)′2 fragment antibodies were purchased from Cell Signaling Technology (Danvers, Mass., USA). Rabbit anti-Histone H3 antibody was purchased from Thermo scientific (Rockford, N.Y., USA). Isotype control IgG (MAB002) and anti-IL-17RB antibodies were purchased from R&D systems (Minneapolis, Minn. USA). Iressa (Gefitinib) and the Src inhibitor AZM475271 were obtained from Tocris Bioscience (R&D systems). EverBrite mounting medium with DAPI was purchased from Biotium (Hayward, Calif. USA).

Cellular Fractionation

Cells (3×10⁵) were seeded in 6-well plates in complete medium for 24 hours then starved for another overnight. Cells were stimulated for 2 hours at 37° C. with IL-17E (10 ng/ml), EGF (long/ml), or a combination of both as indicated. Cells were then washed with PBS and lysed in a lysis buffer (HEPES 10 mM, MgCl2 15 mM KCl 10 mM, DTT 0.5 mM, PMSF 0.2 mM, 1 mM Na₃VO₄, 10 mM NaF, 0.5% Nonidet P-40). After incubation on ice for 20 minutes, the cytoplasmic fraction was obtained after centrifugation 10 seconds at 8000 g 4° C. and nuclear pellet was washed with the lysis buffer. To extract nuclear proteins, the isolated nuclei were suspended in buffer containing HEPES 20 mM, glycerol 25% final concentration, NaCl 420 mM, MgCl2 15 mM and EDTA 0.2 mM completed with PMSF, DTT and Na₃VO₄ as above. After 20 minutes at 4° C. the nuclear extract was collected after centrifugation 10 minutes at 8000 g. Protein concentrations were determined by using Bradford method. Samples were mixed with Laemmli, heated for 10 minutes at 95° C., and then subjected to 8% SDS-PAGE. Proteins were transferred to nitrocellulose membranes, and hybridized with specific anti-EGFR, anti-pY1086EGFR, anti-STAT3 or anti-pY705STAT3 antibodies, then revealed with ECL. Anti-β-actin and anti-Histone H3 antibodies were used as loading control.

Tyrosine Phosphorylation

Cells (3×10⁵) were seeded in 6-well plates in complete medium for 24 hours then starved for another overnight. Cells were then stimulated with IL-17E (10 ng/ml), EGF (10 ng/ml), or a combination of both for 30 min in serum-free medium. Cells were then lysed in 1% Triton X100 buffer and left on ice for 1 hours. Protein samples were then subjected to 8% SDS-PAGE. Western blotting using specific antibodies assessed then the phosphorylation of the various kinases. In some experiments, cells were treated with AZM475271 (10 μM) or Iressa (0.25 μM) prior their stimulation with IL-17E, EGFR, or with the combination of both as indicated.

Immunofluorescence Microscopy

Cells (4.10³) were grown on Lab-Tek chambers slide in their respective complete culture medium for 24 hours then starved for an overnight. Cells were then stimulated with IL-17E (long/ml), EGF (10 ng/ml) or a combination of both for 2 hours at 37° C., washed, and fixed with 4% paraformaldehyde (PFA)-PBS solution at 4° C. Cells were then permeabilized with 0.5% Triton-X-100 in PBS, saturated with 20% FCS in PBS and incubated 18 hours with anti-EGFR (1/500) in 10% FCS-PBS. Slides were mounted with mounting medium containing Dapi then visualized with a Leica DMRB fluorescence microscope and images analysis was performed with the Archimed Software (Microvision).

Results:

IL-17E Phosphorylates EGFR and its Essential Kinases in TNBC Cell Lines

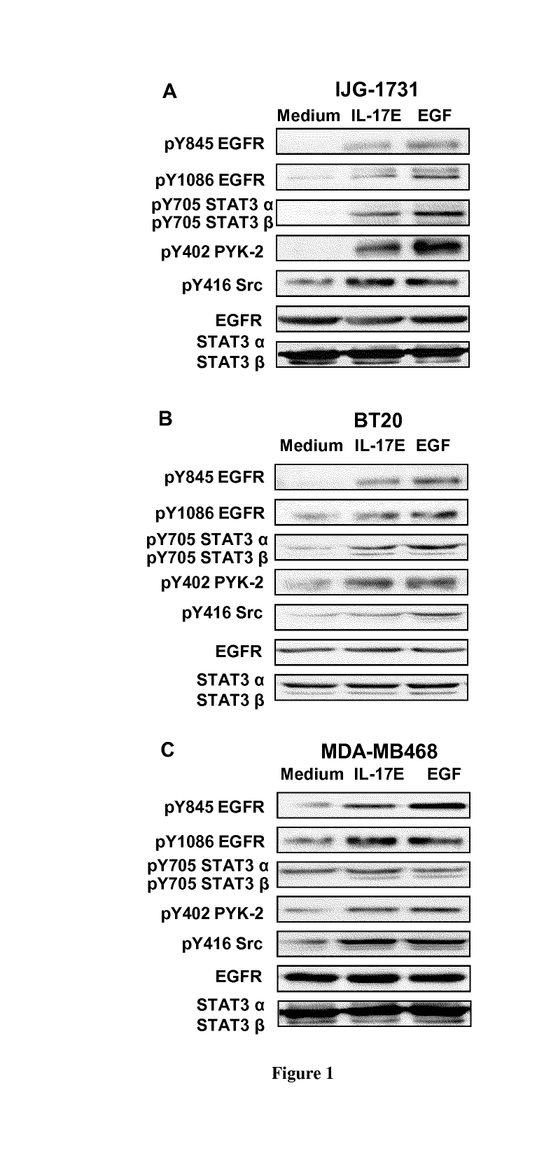

The TNBC ex-vivo-derived IJG-1731 cells, and BT20 and MDA-MB468 are established TNBC tumors cell line models. They express different levels of EGFR that mediate differential EGF-induced phosphorylation patterns (FIG. 1). As such, these tumor cell lines model the heterogeneity of TNBC tumors, and accordingly they were used as an experimental model to investigate IL-17E signaling in TNBC tumors. IJG-1731, BT-20 and MDA-MB468 cells were treated with IL-17E (10 ng/ml) or with EGF (10 ng/ml). Western blot analysis then demonstrated that similar to EGF, both Y845 EGFR, which is a substrate for Src kinase activity, and Y1086 EGFR, which is phosphorylated by the kinase activity of EGFR itself were phosphorylated downstream IL-17E (FIG. 1). The intensity of IL-17E-induced EGFR phosphorylation was comparable to that induced by EGF and in line with cell line basal level of EGFR.

The phosphorylation of STAT3, PYK-2, and Src kinases downstream EGFR is a well-documented essential event of EGFR phosphorylation (Park O K, Schaefer T S and Nathans D. In vitro activation of Stat3 by epidermal growth factor receptor kinase. Proc Natl Acad Sci USA. 1996; 93(24):13704-13708. Shao H, Cheng H Y, Cook R G and Tweardy D J. Identification and characterization of signal transducer and activator of transcription 3 recruitment sites within the epidermal growth factor receptor. Cancer Res. 2003; 63(14):3923-3930). Therefore, we looked at the phosphorylation status of these essential kinases in the three cell lines upon their treatment with IL-17E. Similar to EGF, IL-17E induced considerable phosphorylation of STAT3-α and -β at Y705 in IJG-1731 and BT20 cell lines (FIGS. 1A and B). The phosphorylation level of both STAT3-α and -β was in accordance with the phosphorylation level of Y1086 and Y845 EGFR in these cell lines (FIGS. 1A and B). The IL-17E-induced phosphorylation of STAT3-α and -β was less evident in MDA-MB468 cell line, probably due to elevated STAT3-α constitutive phosphorylation, but likewise consistent with EGFR phosphorylation level. Treatment with IL-17E also induced the phosphorylation of PYK2 and Src kinases at residues Y402 and Y416, respectively (FIG. 1). Compared to EGF, the addition of IL-17E significantly induced or increased the level of pY402PYK-2 as well as pY416Src in the three cell lines. These results indicate that IL-17E and EGF similarly phosphorylate the essential kinases implicated in the phosphorylation of EGFR, and as such IL-17E might contribute to TNBC tumors resistance to EGFR inhibitors.

IL-17E and EGF Signalings are Interconnected

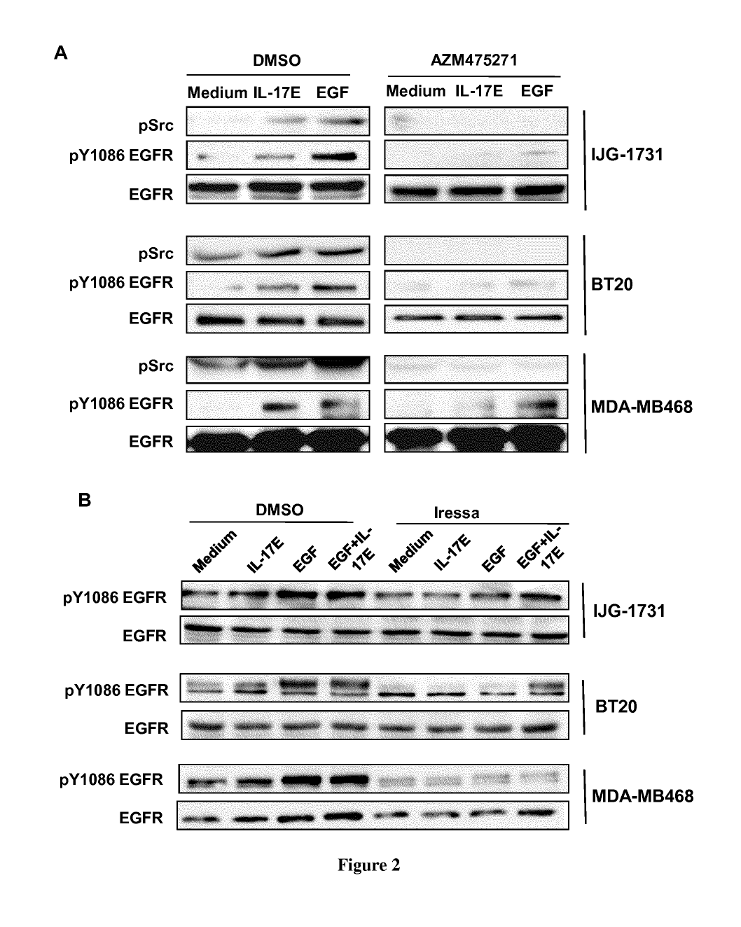

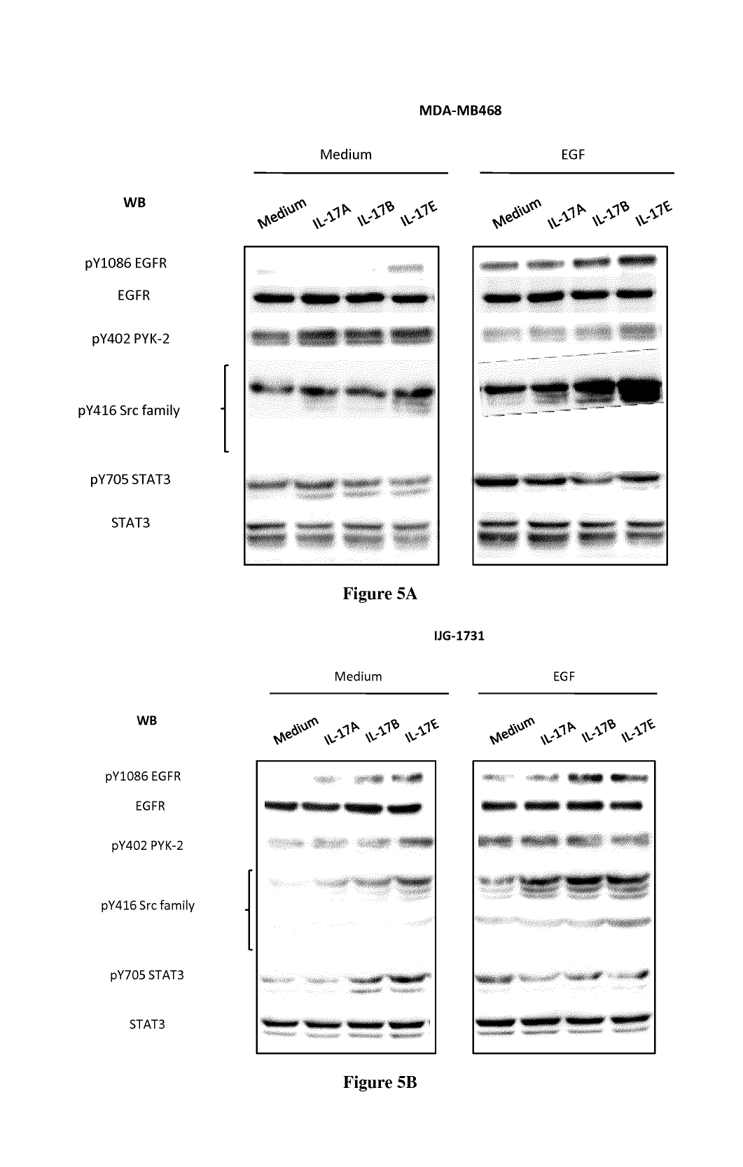

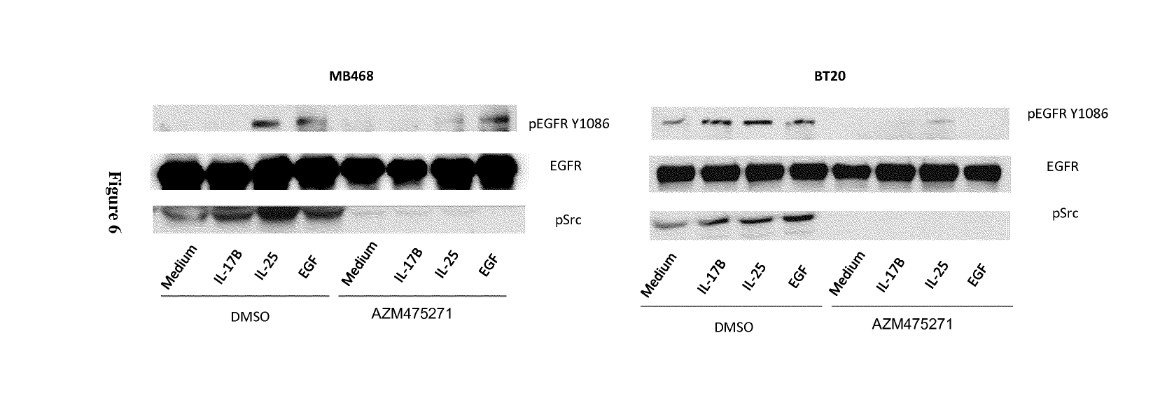

To substantiate the contribution of IL-17E to TNBC tumors resistance to EGFR inhibitors, we examined the interrelation between IL-17E- and EGF-induced signaling. EGFR sustained activity linked to the dimension of TNBC tumors resistance requires both Src and EGFR activation. Therefore, we first determined the involvement of Src kinase in IL-17E-induced EGFR phosphorylation. TNBC tumor cell lines were pre-treated with the Src kinase specific inhibitor AZM475271, then stimulated with either IL-17E or EGF. Treatment with AZM475271 inhibited IL-17E- and EGF-induced Src phosphorylation but also abolished their induced phosphorylation of Y1086 EGFR in IJG-1731 and BT20 cell lines and to a lesser extent in MDA-MB468 (FIG. 2A). Thus, similar to EGF, the IL-17E-induced EGFR phosphorylation is also Src-dependent. This suggests that IL-17E and EGF can trans-activate the EGFR in TNBC tumors. Similar results were obtained with IL-17B in these cell lines (FIG. 5-6).

We then examined whether the IL-17E-induced EGFR phosphorylation requires EGFR activity. TNBC cell lines were treated with EGFR phosphorylation specific inhibitor Iressa then stimulated with IL-17E, EGF or a combination of both and the status of EGFR phosphorylation was analyzed by western blotting. Treatment with Iressa similarly decreased the IL-17E- and EGF-induced Y1086-EGFR phosphorylation in the three cell lines (FIG. 2B). It also remarkably decreased the EGFR tyrosine phosphorylation induced by the combination of IL-17E and EGF in BT20 and MDA-MB468 and to a lesser extends in IJG-1731 (FIG. 2B). Altogether, these data indicate that similar to EGF, IL-17E-induced phosphorylation of EGFR requires both Src and EGFR kinase activities, thus EGF and IL-17E are interconnected and might synergize to activate and sustain EGFR phosphorylation in TNBC tumors.

IL-17E Synergizes with EGF Through its Specific Receptor IL17RA/IL17RB

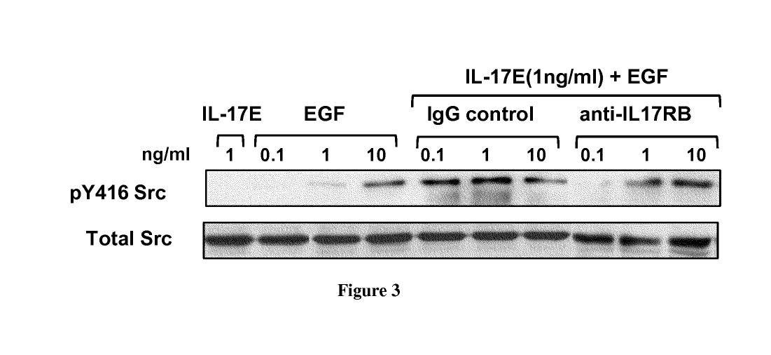

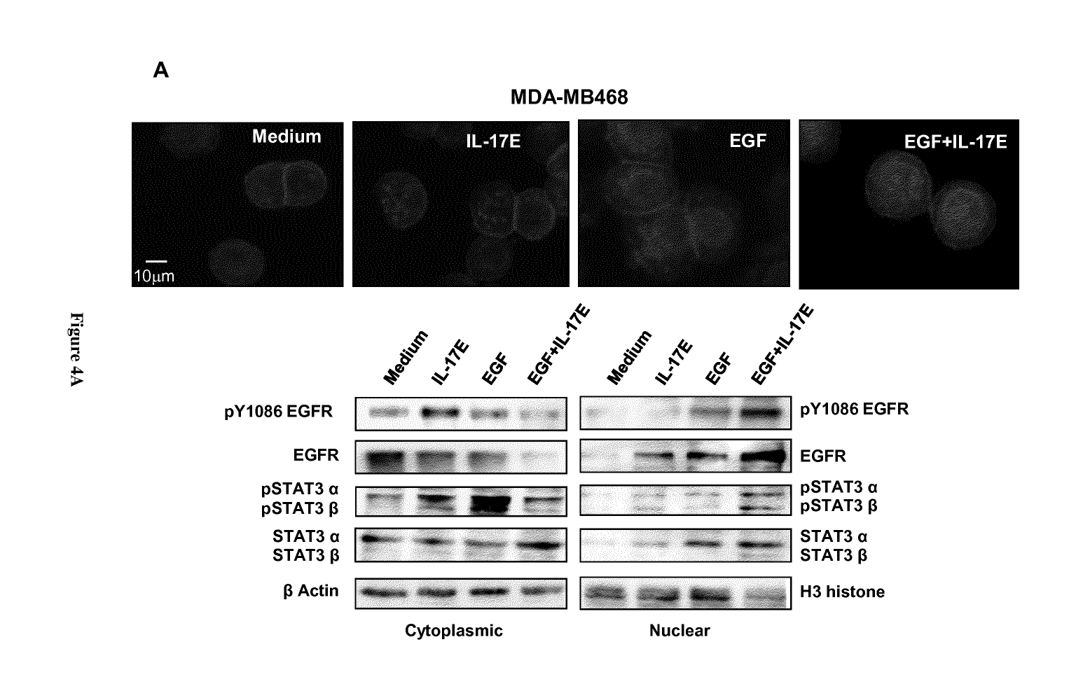

Activation of Src pathway is essential for optimal EGFR activity. Therefore, to explore the synergistic effect of IL-17E and EGF on EGFR activation, we looked at the phosphorylation status of Src in TNBC cells stimulated with increasing concentrations of EGF in the presence or absence of IL-17E. Stimulation of MDA-MB468 with IL-17E or EGF at suboptimal concentrations of 1 ng/ml and 0.1-1 ng/ml, respectively, failed to induce any significant phosphorylation of Src at Y416 (FIG. 3). However, the presence of IL-17E at 1 ng/ml with EGF at suboptimal concentrations of 0.1 and 1 ng/ml promoted the induction of Y416Src phosphorylation to a level similar to that induced by EGF alone at 10 ng/ml (FIG. 3). These results indicate that IL-17E and EGF synergize to activate Src kinase. The presence of anti-IL-17E receptor (IL-17RA/RB)-blocking antibody the anti-IL1RB, but not its isotype control, considerably decreased the phosphorylation of Src at Y416 by EGF at 0.1 and 1 ng/ml in the presence of IL-17E (FIG. 3). Thus, the synergistic effect of IL-17E is mediated by its recruitment to its specific receptor IL-17RA/RB.

IL-17E Contributes to pEGFR and pSTAT3 Translocation to the Nucleus

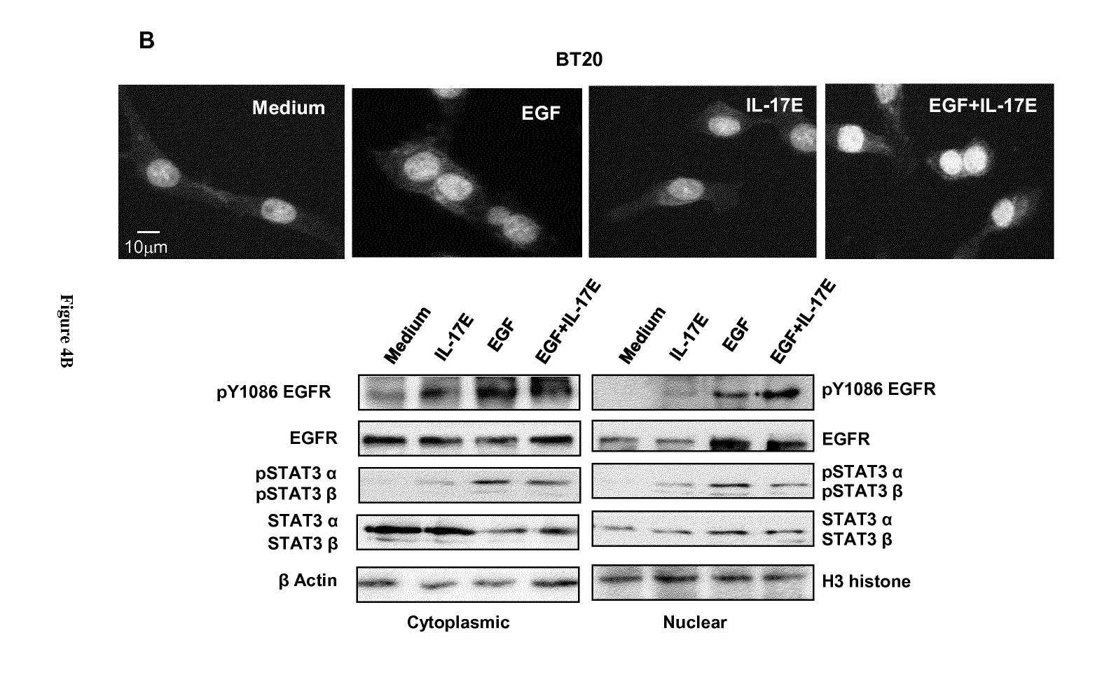

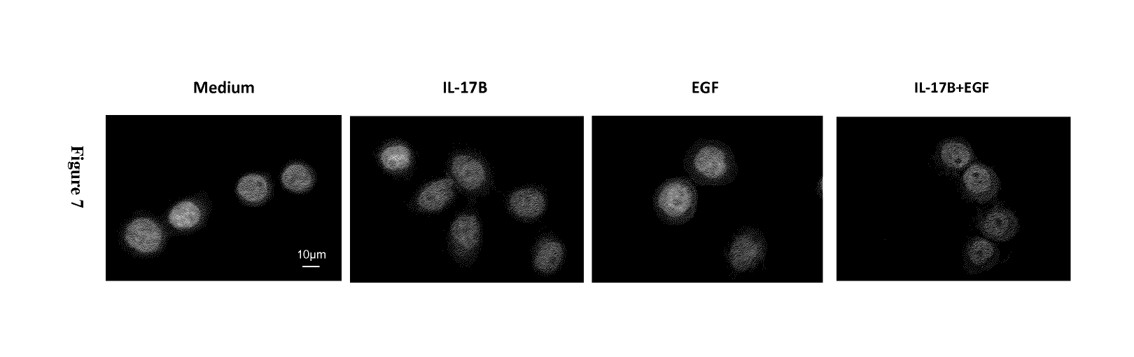

The translocation of phosphorylated EGFR to the nucleus is an integral component of the cascade leading to tumors resistance to EGFR therapeutics. Therefore, to support the contribution of IL-17E to TNBC tumors resistance to this therapy, we investigated the impact of IL-17E on EGFR nuclear translocation. By immunofluorescence microscopy, we examined the localization of EGFR in TNBC cell lines stimulated with EGF, IL-17E, or with a combination of both. In agreement with previous reports, EGF induced strong translocation of EGFR from the membrane to the nucleus in MDA-MB468 TNBC cell line (FIG. 4A). Stimulation with IL-17E also induced the translocation of EGFR to the nucleus but to a lower extends compared to EGF (FIG. 4A). Importantly, the combination of both IL-17E and EGF remarkably increased the translocation induced by each cytokine alone (FIG. 4A). The stimulation with IL-17B on MDA-MB468 cells induced a translocation from the membrane to cytoplasm with a strong perinuclear reorganization (FIG. 7).

To support these data, we then isolated the cytoplasmic and nuclear fractions of IL-17E-, EGF- or IL-17E+EGF-stimulated MDA-MB468 cells and examined the levels of EGFR and its phosphorylated counterpart pY1086EGFR. Compared to EGF alone, IL-17E alone was able to induce the phosphorylation of EGFR but failed to induce its significant translocation to the nucleus despite its capacity to fairly translocate the non-phosphorylated EGFR form. In comparison to EGF and IL-17E alone, almost the totally of EGFR both forms was translocated to the nucleus when MDA-MB468 cells were stimulated with the combination of IL-17E and EGF (FIG. 4A).

STAT3 binds EGFR through a motif including the pY1086. In addition, the correlation between pEGFR and pSTAT3α/β is well established and is implicated in tumor resistance. Therefore, we also looked at the translocation of STAT3 into the nucleus and assessed the status of pSTAT3α and β as well as their non-phosphorylated counterparts. IL-17E or EGF induced the phosphorylation of STAT3α and β but gain it is mainly the combination of both that triggered the most significant translocation of pSTAT3α and β to the nucleus.

To support, we extended these studies to the BT20 cell line. Similar results were obtained. However the EGFR and pSTAT3α and β translocation under the synergetic effect of IL-17E and EGF was less pronounced in BT20 cells compared to that observed with MDA-MB468 cell line (FIG. 4B). This is likely due to the inherent heterogeneity of TNBC tumors in response to various stimuli.

Overall, together these data indicate that the presence of IL-17E or IL-17B within the microenvironment of TNBC tumors would likely promote and sustain the EGFR activation and translocation linked to the dimension of tumor resistance.

### DISCUSSION

Strategies to efficiently combat TNBC are yet to be developed. IL-17B or -E/IL-17RB pathway has been associated with poor prognosis of various types of breast cancer including TNBC. This exploratory study assessed the IL-17E and -B cell signaling pathway as new target suitable for the development of more efficient therapeutic strategies for TNBC. Collectively, our data demonstrate the contribution of such pathway to TNBC resistance to EGFR therapeutics through a loop amplifying and sustaining the phosphorylation of the main EGFR-downstream kinases implicated in this resistance. As such, blocking IL-17B or -E or its receptor in combination with EGFR therapeutics might constitute a novel potential strategy to better treat these tumors.

In agreement with studies performed with hepatocyte growth factor (HGF) in breast cancer (Mueller K L, Hunter L A, Ethier S P and Boerner J L. Met and c-Src cooperate to compensate for loss of epidermal growth factor receptor kinase activity in breast cancer cells. Cancer Res. 2008; 68(9):3314-3322) we found that Src and PYK2 activation related to EGFR phosphorylation could also occur downstream IL-17B or -E receptor in TNBC cells. The inhibition of EGFR activation downstream IL-17E or IL-17B by the Src specific inhibitor AZM475271 further supports this notion. The IL-17B and -E induced Y1086EGFR phosphorylation in TNBC cell lines largely relays on the EGFR kinase activity as evidenced by the specific inhibition of this phosphorylation in the presence of EGFR kinase inhibitor Iressa. However, the phosphorylation of EGFR at Y1086 could also implicate the recruitment of PYK2 in the absence of pEGFR through a pSrc/PYK2. crosstalk (Verma N, Keinan O, Selitrennik M, Karn T, Filipits M and Lev S. PYK2 sustains endosomal-derived receptor signalling and enhances epithelial-to-mesenchymal transition. Nat Commun. 6:6064. Park S Y, Avraham H K and Avraham S. RAFTK/Pyk2 activation is mediated by trans-acting autophosphorylation in a Src-independent manner. J Biol Chem. 2004; 279(32):33315-33322.). The results obtained with IJG-1731 cells in this study support this notion emphasizing the importance of pSrc/PYK2 crosstalk as signaling checkpoint and molecular memory mechanism of tumor metastasis signaling (Park S Y, Avraham H K and Avraham S. RAFTK/Pyk2 activation is mediated by trans-acting autophosphorylation in a Src-independent manner. J Biol Chem. 2004; 279(32):33315-33322.)

The synergistic effect between EGF and IL-17E as well as IL-17B endorses a pro-oncogenic role for IL-17A proteins in TNBC tumors. The presence of such cytokines in the TNBC tumor microenvironment could pre-active the EGFR through its capacity to activate the Src/PYK2 crosstalk. The consequence of such molecular event would be to enhance the sensitivity to EGF and potentially to other EGFR ligands. Very low concentration of EGFR ligands and weak expression (or accessibility) of this receptor would be enough to highly activate tumor cells. The EGFR is an important mediator of tumor development and progression, whereas the IL-17B and -E induce chemoresistance or controls (IL-17E) the cell cycle progression in TNBC as well as in other breast cancer tumors such as the human epidermal growth factor receptor 2 (HER2)-positive tumor cells (Mombelli S, et al. Sci Rep. 5:11874; Emilie Laprevotte, et al. Interleukin-17B promotes chemoresistance of breast tumors through ERK1/2 anti-apoptotic pathway. [abstract]. In: Proceedings of the 106th Annual Meeting of the American Association for Cancer Research; 2015 Apr. 18-22; Philadelphia, Pa. Cancer Res 2015; 75(15 Suppl): Abstract nr 5027. doi:10.1158/1538-7445.AM2015-5027). Whether the IL-17E-induced effects on the cell cycle could be depending on EGFR or its family members (e.g. HER2) transactivation is yet to be defined. Nevertheless, the results herein alongside our previous report showing the importance of IL-17A in the pro-oncogenic signaling in breast cancer (Cochaud S, Giustiniani J, Thomas C, Laprevotte E, Garbar C, Savoye A M, Cure H, Mascaux C, Alberici G, Bonnefoy N, Eliaou J F, Bensussan A and Bastid J. IL-17A is produced by breast cancer TILs and promotes chemoresistance and proliferation through ERK1/2. Sci Rep. 3:3456.) reveal a key role for the presence of IL-17A members within the TNBC microenvironment. They also further expand the concept that inflammation is a critical component of tumour progression (Fort M M, Cheung J, Yen D, Li J, Zurawski S M, Lo S, Menon S, Clifford T, Hunte B, Lesley R, Muchamuel T, Hurst S D, Zurawski G, Leach M W, Gorman D M and Rennick D M. IL-25 induces IL-4, IL-5, and IL-13 and Th2-associated pathologies in vivo. Immunity. 2001; 15(6):985-995; Wang Y H, Angkasekwinai P, Lu N, Voo K S, Arima K, Hanabuchi S, Hippe A, Corrigan C J, Dong C, Homey B, Yao Z, Ying S, Huston D P and Liu Y J. IL-25 augments type 2 immune responses by enhancing the expansion and functions of TSLP-DC-activated Th2 memory cells. J Exp Med. 2007; 204(8):1837-1847.)

In addition to its involvement in tumor progression, our results demonstrate that IL-17E and IL-17B could contribute to the mechanisms of EGFR resistance. We provide the first evidence indicating the involvement of these cytokines in the remodeling and subcellular localization of EGFR. Physiological EGF leads to EGFR degradation through receptor-mediated endocytosis and endosomal trafficking to the lysosomes (Wiley HS. Trafficking of the ErbB receptors and its influence on signaling. Exp Cell Res. 2003; 284(1):78-88.). Therefore, engagement of the IL-17RB co receptor might alter the EGFR degradation process in malignant cells. Importantly, the nuclear fraction of EGFR contributes to the resistance to Cetuximab, (Li C, Iida M, Dunn E F, Ghia A J and Wheeler D L. Nuclear EGFR contributes to acquired resistance to cetuximab. Oncogene. 2009; 28(43):3801-3813.) and also promotes resistance to Iressa (Huang W C, Chen Y J, Li L Y, Wei Y L, Hsu S C, Tsai S L, Chiu P C, Huang W P, Wang Y N, Chen C H, Chang W C, Chen A J, Tsai C H and Hung M C. Nuclear translocation of epidermal growth factor receptor by Akt-dependent phosphorylation enhances breast cancer-resistant protein expression in gefitinib-resistant cells. J Biol Chem. 286(23):20558-20568.).

The IL-17E-translocated nuclear EGFR, which is accompanied by the translocation of pSTAT3, would maintain the phosphorylated status of the translocated pEGFR stressing tumor resistance to anti-EGFR therapeutics. Our data suggest that IL-17E-induced translocation of EGFR could serve as a transporter of pEGFR-associated pSTAT3 to the nucleus (Shao H, Cheng H Y, Cook R G and Tweardy D J. Identification and characterization of signal transducer and activator of transcription 3 recruitment sites within the epidermal growth factor receptor. Cancer Res. 2003; 63(14):3923-3930; Lin S Y, Makino K, Xia W, Matin A, Wen Y, Kwong K Y, Bourguignon L and Hung M C. Nuclear localization of EGF receptor and its potential new role as a transcription factor. Nat Cell Biol. 2001; 3(9):802-808.). In the nucleus, Stat3 can activate the transcription of genes involved in tumor metastasis as well as the transcription of anti-apoptotic and angiogeneic genes similar to its functioning in various types of cancer (Bromberg J F, Wrzeszczynska M H, Devgan G, Zhao Y, Pestell R G, Albanese C and Darnell J E, Jr. Stat3 as an oncogene. Cell. 1999; 98(3):295-303; Niu G, Wright K L, Huang M, Song L, Haura E, Turkson J, Zhang S, Wang T, Sinibaldi D, Coppola D, Heller R, Ellis L M, Karras J, Bromberg J, Pardoll D, Jove R, et al. Constitutive Stat3 activity up-regulates VEGF expression and tumor angiogenesis; Lo H W, Hsu S C, Ali-Seyed M, Gunduz M, Xia W, Wei Y, Bartholomeusz G, Shih J Y and Hung M C. Nuclear interaction of EGFR and STAT3 in the activation of the iNOS/NO pathway. Cancer Cell. 2005; 7(6):575-589.

The IL-17B or -E-induced signaling is mediated through its specific homodimer IL-17RB or heterodimer receptor IL17RA/IL17RB. Thus, IL-17-induced resistance to anti-EGFR treatments conferred by the activation of Src, STAT3 and the EGFR translocation, are at least in part, under the control of IL-17RB co receptor. Further investigation of these signaling mechanisms could improve the specificity and may be the efficacy of biotherapies targeting these receptors.

Our findings advance the current understanding of anti-EGFR immunotherapies failure in breast cancer. IL-17B and -E-induced signaling might also be interconnected with other members of the EGF receptors family such as HER2 and HER3 and contribute to their resistance to drugs. Furthermore, these cytokines might promote resistance of IL-17RA/RB-expressing tumors such as head and neck, non-small cell lung, pancreatic and colorectal cancers to anti-EGFR drugs. IL-17E is abundant in most of the metastatic tissues in brain (Sonobe Y, Takeuchi H, Kataoka K, Li H, Jin S, Mimuro M, Hashizume Y, Sano Y, Kanda T, Mizuno T and Suzumura A. Interleukin-25 expressed by brain capillary endothelial cells maintains blood-brain barrier function in a protein kinase Cepsilon-dependent manner. J Biol Chem. 2009; 284(46):31834-31842); liver (Wang A J, Yang Z, Grinchuk V, Smith A, Qin B, Lu N, Wang D, Wang H, Ramalingam T R, Wynn T A, Urban J F, Jr., Shea-Donohue T and Zhao A. IL-25 or IL-17E Protects against High-Fat Diet-Induced Hepatic Steatosis in Mice Dependent upon IL-13 Activation of STATE. J Immunol. 195(10):4771-4780); and lung (Yao X, Sun Y and Wang W. Interleukin (IL)-25: Pleiotropic roles in asthma. Respirology. 10.1111/resp.12707) which further emphasize the importance of advancing the knowledge concerning the IL-17E and tumor progression. Concerning IL-17B, this cytokine is abundant in pancreatic and breast cancer (Wu H H, Hwang-Verslues W W, Lee W H, Huang C K, Wei P C, Chen C L, Shew J Y, Lee E Y, Jeng Y M, Tien Y W, Ma C, Lee W H. Targeting IL-17B-IL-17RB signaling with an anti-IL-17RB antibody blocks pancreatic cancer metastasis by silencing multiple chemokines. J Exp Med. 2015 Mar. 9; 212(3):333-49; Huang C K, Yang C Y, Jeng Y M, Chen C L, Wu H H, Chang Y C, Ma C, Kuo W H, Chang K J, Shew J Y, Lee W H. Autocrine/paracrine mechanism of interleukin-17B receptor promotes breast tumorigenesis through NF-κB-mediated antiapoptotic pathway. Oncogene. 2014 Jun. 5; 33(23):2968-77).

In summary, our studies provide in the context of TNBC first evidence indicating the critical role of IL-17E as well as IL-17B in tumor resistance to anti-EGFR therapeutics. Blocking either IL-17E, IL-17B or the receptor in combination of current therapeutics would reduce the likelihood of tumor resistance enhancing therapeutic efficiency.

### REFERENCES

Throughout this application, various references describe the state of the art to which this invention pertains. The disclosures of these references are hereby incorporated by reference into the present disclosure.

1-15. (canceled)

16. A composition comprising an HER inhibitor and at least one of an IL-17B inhibitor or IL-17E inhibitor.

17. The composition according to claim 16, wherein the HER inhibitor is an EGFR (HER1) inhibitor.

18. The composition according to claim 16, wherein the HER inhibitor is a tyrosine kinase inhibitor.

19. The composition according to claim 16, wherein the HER inhibitor is selected from the group consisting of cetuximab, panitumumab, zalutumumab, nimotuzumab, erlotinib, gefitinib, lapatinib, lapatinib ditosylate, neratinib, canertinib, vandetanib, afatinib, TAK-285, ARRY334543, Dacomitinib, OSI-420 (Desmethyl Erlotinib), AZD8931), AEE788 (NVP-AEE788), Pelitinib (EKB-569), CUDC-101, XL647, BMS-599626 (AC480), PKC412, BIBX1382, AP261 13, and combinations thereof.

20. The composition according to claim 16, comprising an IL-17B inhibitor selected from the group consisting of an antibody directed against IL-17B or an antibody directed against a receptor of IL-17B.

21. The composition according to claim 16, comprising an IL-17E inhibitor selected from the group consisting of an antibody directed against IL-17E or an antibody directed against a receptor of IL-17E.

22. The composition according to claim 21, comprising an IL-17E inhibitor selected from the group consisting of an antibody directed against IL-17E.

23. The composition according to claim 21, wherein the IL-17E inhibitor binds IL-17RA, IL-17RB, or a dimeric complex thereof.

24. The composition according to claim 16, wherein the HER inhibitor is an inhibitor of HER expression, the IL-17B inhibitor is an inhibitor of IL-17B expression, or the IL-17E inhibitor is an inhibitor of IL-17E expression.

25. The composition according to claim 16, wherein at least one of the HER inhibitor, IL-17B inhibitor, or IL-17E inhibitor is an siRNA or an antisense oligonucleotide.

26. A method of treating cancer, comprising administering to a patient in need thereof an HER inhibitor and at least one of an IL-17B inhibitor or IL-17E inhibitor.

27. The method according to claim 26, wherein the cancer is resistant to HER inhibitors.

28. The method according to claim 26, wherein the cancer is resistant to EGFR (HER1) inhibitors.

29. The method according to claim 26, wherein the cancer is selected from the group consisting of colorectal cancer, non-small cell lung carcinoma (NSCLC), adrenocortical carcinoma (ACC), pancreatic cancer, head and neck cancer, breast cancer, or neuroblastoma.

30. The method according to claim 26, wherein the cancer is selected from triple negative breast cancer.

31. The method according to claim 26, wherein the HER inhibitor is administered at a reduced dosage level relative to therapeutic regimens in which an IL-17B inhibitor or IL-17E inhibitor is not also provided.
